(19) United States
(12) Patent Application Publication
(10) Pub. No.: US 2024/0091019 A1
(43) Pub. Date: Mar. 21, 2024

(54) KNEE JOINT IMPLANT AUGMENTATION AND KNEE JOINT IMPLANT

(71) Applicant: Aesculap AG, Tuttlingen (DE)

(72) Inventors: Martin Hirt, Stockach (DE); Arthur Bollinger, Tuttlingen (DE)

(21) Appl. No.: 18/470,743

(22) Filed: Sep. 20, 2023

(30) Foreign Application Priority Data
Sep. 21, 2022 (DE) 10 2022 124 193.9

(51) Int. Cl. A61F 2/38

(52) U.S. Cl. CPC A61F 2/389; A61F 2/3859; A61F 2002/30736

(57) ABSTRACT

A knee joint implant augmentation configured to be arranged between a knee joint implant and a tibia in an implanted state includes a first contact surface which, in the implanted state, is configured to rest at least partially flat on the knee joint implant, a second contact surface lying opposite the first contact surface and configured to rest at least partially flat on the tibia in the implanted state, and a lateral surface, which connects a rim of the first contact surface to a rim of the second contact surface. The lateral surface is configured such that a portion of the lateral surface, which is arranged in a patellar position in the implanted state of the knee joint implant augmentation, is angled at a closed angle greater than 0° with respect to an axis perpendicular to the first contact surface.

### CROSS-REFERENCE TO RELATED APPLICATION(S)

This application claims priority under 35 U.S.C. § 119 to German Application No. 10 2022 124 193.9, filed Sep. 21, 2022, the content of which is incorporated by reference herein in its entirety.

### FIELD

The present disclosure relates to a knee joint implant augmentation for a knee joint implant and a knee joint implant.

### BACKGROUND

Knee joint prostheses or implants or knee endoprostheses are used to replace the knee joint, which is usually destroyed by knee arthrosis. The knee joint is partially or completely replaced by the knee joint prostheses or implants.

Known knee joint implants have a femur element, a tibia element and an inlay between the femur element and the tibia element. The inlay is a sliding surface over which the femur element can slide on the tibia element. The femur element has a shaft to connect it firmly to the femur. The tibia element has a tibia shaft to connect the tibia element firmly to the tibia. If a large part of the tibia is removed, the thin tibia element cannot replace the lost bone. Therefore, a so-called knee joint implant augmentation or knee implant augmentation is also used. The knee joint implant augmentation is essentially a (flat) plate that acts as a spacer between the cut tibia and the tibia element of the knee joint implant.

Such a knee joint implant augmentation is known, for example, from U.S. Pat. No. 11,266,509 B2. In this case, the knee joint implant augmentation is anatomically configured in such a way that it is adapted to the shape of the cut bone. A distal or lower surface of the knee joint implant augmentation is thereby substantially adapted to a cross-sectional shape of the truncated tibia. An area of the distal surface is smaller than an area of a proximal surface of the knee joint implant augmentation. EP 1 360 950 B1 also discloses such a knee joint implant augmentation.

In the case of known knee joint implant augmentation, the entire tibia element has to be shifted in the anterior direction or forward toward the knee during attachment in order to ensure good support of the tibia element with the knee joint implant augmentation on the tibia. In this part, the tibia expands in the anterior direction.

However, this shift in the anterior direction is disadvantageous. On the one hand, it results in an undesirable protrusion of the tibia element over the bone. On the other hand, the repositioning during insertion of the implant means that the reference to a medullary canal of the bone is lost. In revision surgeries, shafts are often used, which should optimally follow the medullary canal of the bone/tibia. The medullary canal thus forms a mechanical axis of the bone/tibia. By moving it forward, the tibia element and in particular the shaft of the tibia element is no longer aligned along the medullary canal/mechanical axis of the tibia. The error due to repositioning can only be corrected by adjustable shafts. However, most shafts are only adjustable medial-laterally and not anterior-posterior. Therefore, positioning errors of the tibia may occur. Thus, known knee joint implants may be used inappropriately.

More specifically, a conflicting goal may occur when placing the implant. If the tibial shaft is aligned with the medullary canal of the tibia, the bearing surface between the implant and especially the knee implant augment will not match the truncated tibia. However, if the augment is aligned with the shape of the truncated tibia, the tibial shaft may deviate from the mechanical axis of the medullary canal. This trade-off could be resolved by implants with adjustable shafts. However, most shafts are only medial-lateral adjustable. Furthermore, adjusting the shafts during surgery is disadvantageous.

### SUMMARY

Thus, the object of the present disclosure is to overcome or at least reduce the disadvantages of the prior art, in particular to provide a knee joint implant augmentation by which a knee joint implant can be optimally attached to the tibia. It is further intended to provide a knee joint implant that can be optimally connected to the tibia.

The present disclosure relates to a knee joint implant augmentation or a spacer for a knee joint implant, which is prepared and configured to be arranged between a knee joint implant and a tibia in an implanted state. The knee joint implant augmentation has a first contact surface, which, in the implanted state, is configured to rest at least partially flat on the knee joint implant, a second contact surface lying opposite the first contact surface and configured to be able to rest at least partially flat on the tibia in the implanted state, and a lateral surface or peripheral surface, which connects a rim of the first contact surface to a rim of the second contact surface. The lateral surface is configured such that a portion of the lateral surface/a lateral-surface portion, which is arranged in a patellar position in the implanted state of the knee joint implant augmentation, is angled starting from the first contact surface in the patellar direction at a closed angle greater than 0° with respect to an axis perpendicular to the first contact surface.

The knee joint implant replaces the original knee joint at least partially and is thus inserted or operated into the body in a positionally determined manner. The knee joint implant augmentation is positionally determined on the knee joint implant and is thus also positionally determined with respect to the knee joint or the body.

Positionally determined in this context means that the position and orientation of the knee joint implant augmentation with respect to the knee joint implant is defined by suitable means on the knee joint implant augmentation or on the knee joint implant or by an interaction of means on both components. Thus, in particular, it is defined in which direction the individual portions of the lateral surface point when the entire knee joint implant is inserted or implanted (as intended).

In this context, the patellar direction is to be understood as the direction that points forward in humans, in particular toward the kneecap. The patellar alignment is to be understood as the anterior direction of the body.

The first contact surface or proximal surface in this context is the side of the knee joint implant augmentation that points upward in humans, i.e. toward the upper body or respectively from the knee toward the thigh. The second contact surface or caudal or distal side is the side of the knee joint implant augmentation facing the foot or lower leg. The first contact surface is therefore in contact with the knee joint implant during assembly, and the second contact surface is in contact with the tibia.

The tibia extends along its longitudinal axis. The medullary canal essentially follows the longitudinal axis of the tibia. The patellar lateral-surface portion is angled with respect to the longitudinal axis of the tibia at the closed angle not equal to 0°.

In other words, the knee joint implant augmentation is an augmentation or spacer for a knee joint implant having a preferably flat, approximately u-shaped or horseshoe-shaped base plate having a first (base) surface, a second (base) surface facing the first (base) surface, and a lateral or peripheral surface connecting and bounding the first (base) surface and second (base) surface. The lateral surface has a portion that faces in the patellar or anterior direction when the knee joint implant augmentation is positionally determined to abut the knee joint implant and, more specifically, a distal surface of a tibia element of the knee joint implant.

The knee joint implant augmentation thus has a protruding nose at the patellar lateral-surface portion, the nose preferably being formed at the distal side of the lateral-surface portion. Overall, the anterior portion is formed and adapted such that the second contact surface of the knee joint implant augmentation at least partially corresponds to the shape of the (truncated) tibia.

The axis perpendicular to the first contact surface may also be arranged perpendicular to the second contact surface. The angled patellar lateral-surface portion may span a nose that extends over a base area of the first contact surface. The axis may be a normal vector to the first contact surface. Since the first contact surface and the second contact surface are preferably parallel to each other, the axis may also be a normal vector to the first contact surface. In the implanted state of the knee joint implant augmentation, the axis may be aligned parallel to or correspond to the longitudinal axis of the medullary canal of the tibia.

Due to the knee joint implant augmentation according to the disclosure, the knee joint implant is optimally adapted to the shape of the tibia. As a result, the knee joint implant augmentation adheres better on the tibia, or ensuring the support of the tibia element of the knee joint implant with the knee joint implant augmentation on the tibia is improved. In particular, the knee joint implant augmentation has a large support surface on the truncated tibia. Thus, the tibia element does not need to be moved in the anterior/patellar direction during surgery to increase the support surface between the knee joint implant augmentation and the tibia, and can therefore remain in the optimal position. In the optimal position, a shaft of the tibia element is aligned with respect to the medullary canal of the tibia. The tibia element can thus be better secured to the tibia and positioning errors of the tibia can be avoided. In particular, the tibia element is aligned along the mechanical axis or medullary canal of the tibia.

Furthermore, the manufacture of the knee joint implant augmentation according to the disclosure is simple and inexpensive.

The object of the present disclosure is further solved by a knee joint prosthesis/knee joint implant. The knee joint implant comprises a femur element or implant and a tibia element or implant. The tibia element comprises a base plate, a shaft extending distally from the base plate and adapted to be inserted into the tibia, and preferably a (reinforcement) wing extending between the base plate and the shaft. Furthermore, the knee joint implant may preferably comprise an inlay/sliding surface/insert that is placed between the femur element and the tibia element and allows the two elements to slide on each other. The knee joint implant further comprises a knee joint implant augmentation having a first contact surface, a second contact surface opposite the first contact surface, and a lateral surface, which connects a rim of the first contact surface to a rim of the second contact surface. The first contact surface rests at least partially flat on the base plate. The second contact surface is configured to rest at least partially flat on the bone. The lateral surface is configured such that a portion of the lateral surface, which, in the implanted state of the joint implant is arranged in a patellar position, is angled at a closed angle greater than 0° with respect to a longitudinal axis of the shaft.

In the implanted state of the knee joint implant, the shaft of the tibia element extends distally from the base plate. In the implanted state, the shaft is configured to be recessed/inserted into the tibia. The longitudinal axis may extend along the longitudinal axis of the medullary canal of the tibia.

The knee joint implant can be particularly firmly connected to the bone by the angled patellar lateral-surface portion and is easy to insert or operate on, since the support surface of the knee joint implant on the tibia is particularly large. In particular, the knee joint implant and especially the tibia element do not have to be moved forward during insertion. The knee joint implant is therefore aligned with the longitudinal axis of the tibia and the longitudinal axis of the medullary canal during implantation.

The patellar lateral-surface portion in the implanted state of the joint implant can also be angled with respect to the longitudinal axis of the medullary canal of the tibia in the closed angle.

The knee joint implant is preferably positioned between a distal portion of the femur and a proximal portion of the tibia. The femur element can be connected to the femur and the tibia element to the tibia. In particular, the partial implants can be connected in particular to the corresponding bones via the respective shafts.

According to an optional aspect of the present disclosure, the patellar lateral-surface portion is angled at the closed angle not equal to 0° with respect to an axis perpendicular to the first contact surface and/or the second contact surface. The axis may be a normal vector to the first contact surface. Since the first contact surface and the second contact surface are preferably parallel to each other, the axis may also be a normal vector to the first contact surface. In the implanted state of the knee joint implant augmentation, the axis may be aligned parallel to or correspond to the longitudinal axis of the medullary canal of the tibia. The angled patellar lateral-surface portion may span a nose that extends over a base area of the first contact surface.

The knee joint implant augmentation may have approximately the shape of an (oblique) cylinder or a truncated cone. The axis may be a longitudinal axis of the cylinder and/or of the truncated cone.

An angle between the first contact surface and the patellar lateral-surface portion may be greater than 90°, such that a nose spanned by the angled patellar lateral-surface portion extends over a base area of the first contact surface. Thus, the angle of the patellar lateral-surface portion opens toward the distal direction. The area of the second contact surface can thus be larger than the area of the first contact surface. Thus, the second contact surface is at least partially adapted to a cross-section of the (truncated) tibia. The.

An angle between the second contact surface and the patellar lateral-surface portion may therefore also be not equal to 90°. Particularly preferably, the angle between the second contact surface and the patellar lateral-surface portion in the implanted state of the knee joint implant augmentation is less than 90°.

Preferably, at least three sides of the lateral surface are angled with respect to the first and/or the second contact surface. I.e. at least three sides of the lateral surface are not aligned perpendicular to the contact surfaces, but are angled at an angle not equal to 90° the contact surfaces.

According to another optional aspect of the present disclosure, the closed angle between the patellar lateral-surface portion and the axis opens in a caudal/distal direction. That is, the surface area of the second contact surface can be increased. This can ensure that (the truncated) tibia and the knee joint implant augmentation overlap over as large an area as possible and ensure a firm connection.

Preferably, the knee joint implant augmentation is approximately u-shaped or horseshoe-shaped. The patellar lateral-surface portion may be positioned in a leg/arm of the horseshoe. The u-shaped or horseshoe-shaped knee joint implant augmentation may have two legs/arms extending substantially parallel from a central portion in a lateral direction. The patellar lateral-surface portion may be positioned at one of the two legs. The patellar lateral-surface portion may be positioned to face an outer surface of the ‘horseshoe’.

In the implanted state of the knee joint implant augmentation, the central portion (of the ‘horseshoe’) may point in the lateral direction of the knee joint. The two legs may extend in the lateral direction. Thus, in the implanted state, one leg may be positioned in a patellar or anterior position and the other in a posterior position. In particular, the patellar lateral-surface portion may be positioned at the patellar leg of the horseshoe-shaped knee joint implant augmentation. Thus, the patellar lateral-surface portion can increase the patellar support surface between the knee joint implant augmentation and the tibia and can allow the knee joint implant to be mounted aligned with the longitudinal axis of the tibia.

According to another optional aspect of the present disclosure, an elongated notch in the (u-shaped or horseshoe-shaped) knee joint implant augmentation is prepared and configured to receive a (reinforcement) wing of the knee joint implant, and the patellar lateral-surface portion extends substantially parallel to a longitudinal extent of the notch. The (reinforcement) wing may be inserted into the notch of the u-shaped or horseshoe-shaped knee joint implant augmentation. The combination of the orientation of the (reinforcement) wing and the notch can attach the knee joint implant augmentation to the knee joint implant in the implanted state in a positionally determined manner. Thus, the notch can define the position and orientation of the knee joint implant augmentation on the knee joint implant.

Preferably, the knee joint implant augmentation has the shape of a parallelogram in a side view or in a cross section. Both the patellar lateral-surface portion and the posterior lateral-surface portion may be angled. In particular, the two lateral-surface portions may be angled parallel to each other.

It is possible that the two lateral-surface portions are each angled with respect to the axis, but not angled at the same angle.

Preferably, at least part of the first base surface of the knee joint implant augmentation is adapted to the shape of the base plate of the tibia element. I.e. the surface of the knee joint implant augmentation that directly abuts the base plate of the tibia element has at least partially the shape of the base structure. The two elements thus merge seamlessly.

According to another optional aspect of the present disclosure, a portion of the second base surface of the knee joint implant augmentation is formed and adapted to at least correspond to an outer contour of the tibia. That is, the second base surface may substantially conform to the shape of the cross-section of the tibia. Thus, the truncated tibia may transition into the knee joint implant augmentation without a shoulder. That is, the cross-section of the truncated tibia may correspond at least in part or portion to the second base surface.

### BRIEF DESCRIPTION OF THE DRAWINGS

FIG. 1 shows a knee joint implant according to the prior art;

FIG. 2 shows a perspective view of a tibia element with a knee joint implant augmentation according to the prior art on a tibia;

FIG. 3 shows a side view of the knee joint implant augmentation according to the prior art;

FIG. 4 shows a side view of a knee joint implant augmentation according to the present disclosure;

FIG. 5 shows an isometric drawing of the knee joint implant augmentation according to the present disclosure;

FIG. 6 shows a bottom side of the knee joint implant augmentation according to the present disclosure; and

FIG. 7 shows a side view of a tibia element with the knee joint implant augmentation according to the present disclosure.

### DETAILED DESCRIPTION

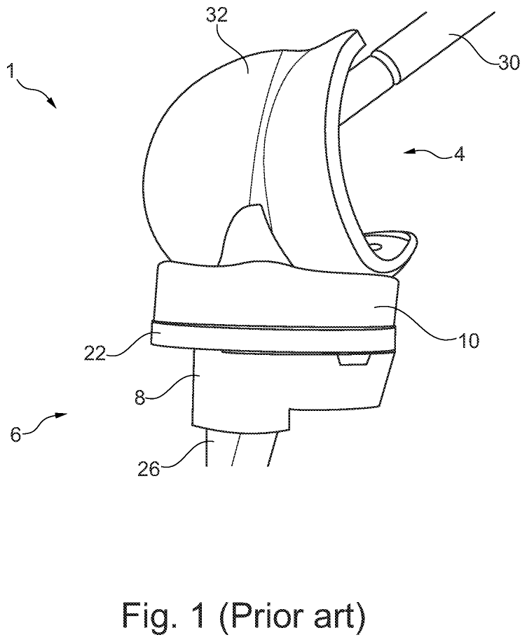

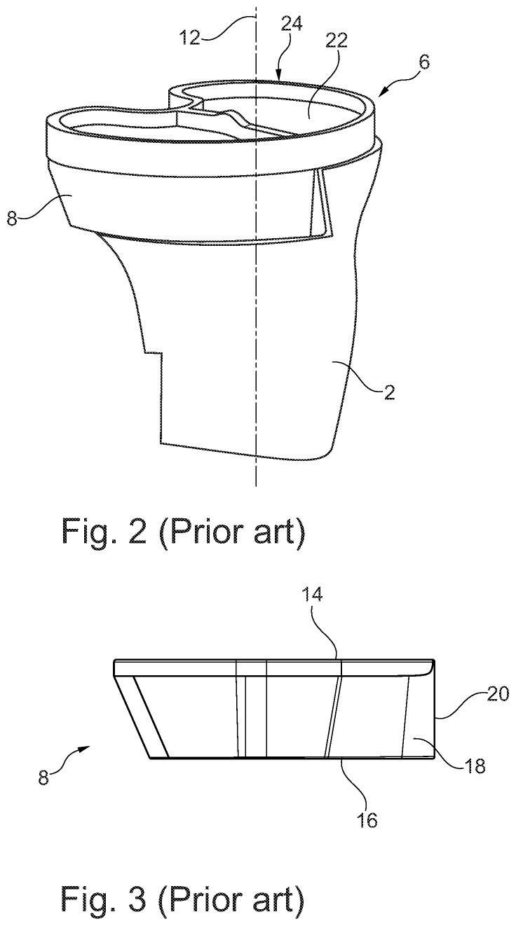

FIGS. 1 to 3 show a knee joint implant or knee prosthesis 1 according to the prior art. In an implanted state, the knee joint implant 1 is preferably positioned between a distal portion of the femur (thigh bone) and a proximal portion of the tibia (shin bone) 2 and has a femur element or femur implant 4, a tibia element or tibia implant 6 with a (knee joint implant) augmentation 8 and an inlay/sliding layer 10. The inlay 10 is arranged between the femur element 4 and the tibia element 6 and allows the femur element 4 to slide relative to the tibia element 6.

The knee joint implant augmentation 8 is configured to be arranged between the knee joint implant 1 and the tibia 2 in the implanted state and has a proximal first contact surface 14, which is configured to rest at least partially flat on the knee joint implant 1 in the implanted state of the knee joint implant augmentation 8, and a distal/caudal second contact surface 16, which is configured to rest at least partially flat on the tibia 2 in the implanted state. The knee joint implant augmentation 8 further comprises a lateral surface 18 connecting a rim of the first contact surface 14 to a rim of the second contact surface 16. The lateral surface 18 has a lateral-surface portion 20 that is arranged at a patellar position in the implanted state of the knee joint implant augmentation 8.

A thin base plate 22 of the tibia element 6 lies against the first contact surface 14. The base plate 22 has a receiving structure 24 for the inlay 10 on its proximal side. On the caudal/distal side of the base plate 22, a tibia shaft 26 extends in the distal direction. The tibia shaft 26 is prepared and adapted to be countersunk into the tibia 2 to ensure a firm connection between the tibia 2 and the tibia element 6. The shaft 26 corresponds to a longitudinal axis 12 of a medullary canal (not shown) of the tibia 2 in the implanted state of the knee joint implant 1.

If a large part of the tibia 2 is removed, the thin tibia element 6 cannot replace the lost bone. Therefore, the knee joint implant augmentation 8 is used in addition. The first contact surface 14 of the knee joint implant augmentation 8 is prepared and adapted to abut a distal surface of the base plate 22.

The femur element 4 is located on the side of the inlay 10 opposite the tibia element 6. The femur element 4 has a connection portion 32, with which the femur element 4 is connected to the femur, and a roll portion 34, which rolls on the inlay 10. The inlay 10 allows the femur element 4 and the tibia element 6 to slide on top of each other/to roll on each other and in a sense acts as a bearing between the two elements 4, 6. The connection portion 32 is essentially an elongated shaft that is connected to the femur. The roll portion 34 has a convex semicircular curve and is attached to the connection portion 32.

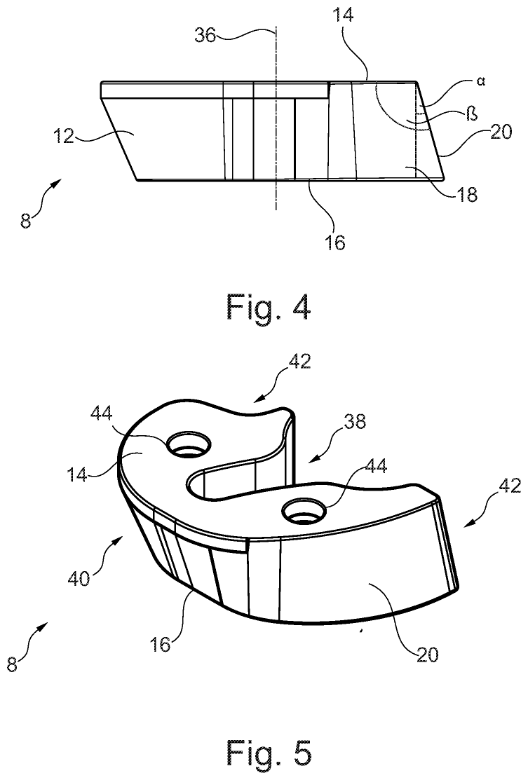

FIG. 4 shows a side view of the knee joint implant augmentation 8 according to the disclosure. The knee joint implant augmentation 8 has the first contact surface 14, the second contact surface 16 facing the first contact surface 14, and the lateral surface 18 connecting the rim of the first contact surface 14 to the rim of the second contact surface 16. Furthermore, the knee joint implant augmentation 8 has an axis 36 that is perpendicular to the first contact surface 14 and/or the second contact surface 16. The lateral-surface portion 20, which is arranged in a patellar position in the implanted state of the knee joint implant augmentation 8, is angled at a closed angle a not equal to 0° with respect to the longitudinal axis 12 of the medullary canal of the tibia 2. Angle β between the first contact surface 14 and the patellar lateral-surface portion 20 is greater than 90°.

I.e. an angle between the axis 36 of the knee joint implant augmentation 8 and the patellar lateral-surface portion 20 is preferably greater than 0°. The axis 36 is a normal vector to the first contact surface 14 and the second contact surface 16, respectively. The patellar lateral-surface portion 20 thus increases a support surface between the tibia 2 and the second contact surface 16 compared to the prior art. This allows for a better connection of the knee joint implant augmentation 8 with the tibia 2. Another portion of the lateral surface 18 opposite the angled patellar lateral-surface portion 20 is also angled. The two angled portions may have the same closed angle.

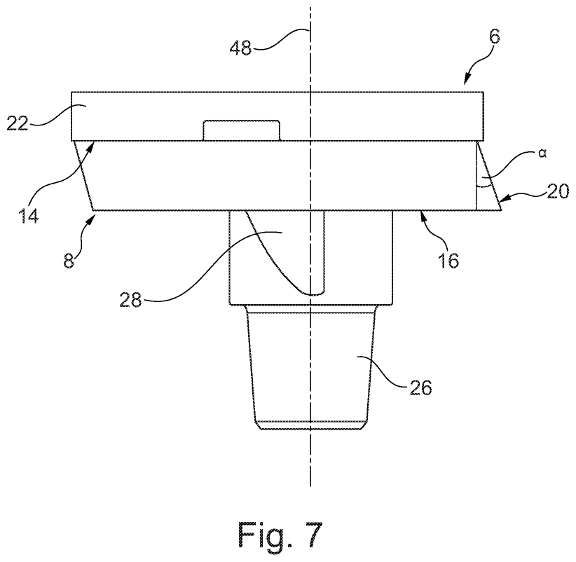

FIG. 5 shows an isometric view of the knee joint implant augmentation 8 according to the present disclosure. The knee joint implant augmentation 8 does not have a continuous circular or oval surface or plate, but is approximately u-shaped or horseshoe-shaped. The knee joint implant augmentation 8 has an elongated notch 38 adapted and prepared to receive the tibia shaft 26 and possibly the (reinforcement) wing 28 (shown in FIG. 7). The knee joint implant augmentation 8 has a central portion 40 and two legs 42 extending laterally from the central portion along the notch 38 in the implanted state. The angled patellar lateral-surface portion 20 is formed on the leg 42 that is arranged at a patellar position in the implanted state.

The lateral surface 18, at least in portions, is not oriented perpendicular to the two contact surfaces 14 and 16. The portions of the lateral surface 18, which are positioned in the central portion 40 of the u-shaped knee joint implant augmentation 8 and on a posterior front side, are angled with respect to the longitudinal axis 36 in the implanted state of the knee joint implant augmentation 8. Thereby, the first contact surface 14 protrudes in sections beyond the second contact surface 16. Thus, the shape of the knee joint implant augmentation 8 is substantially adapted to the shape of the tibia 2. The knee joint implant augmentation 8 also has two holes 44 through the base plate 12, through which the knee joint implant augmentation 8 is screwed to the tibia element 6.

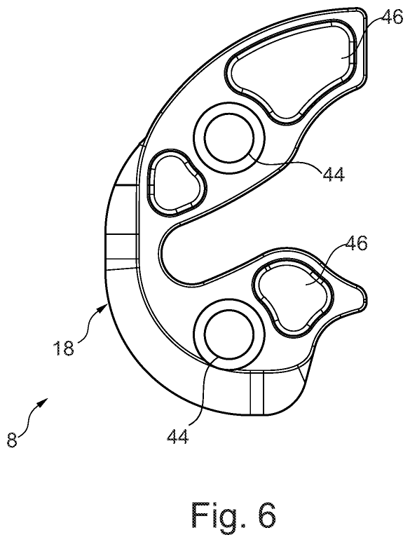

FIG. 6 shows an underside of the knee joint implant augmentation 8. The holes 44 are continuous through the entire thickness of the knee joint implant augmentation 8 and open into the second contact surface 16. The underside or second contact surface 16 of the knee joint implant augmentation 8 has a number of recesses 46. The recesses 46 are adapted to precisely fit the knee joint implant augmentation 8 to the tibia 2. In the bottom view, it is shown that the lateral surface 18 protrudes over the second contact surface 16 in a lateral and a posterior portion.

FIG. 7 shows a side view of the tibia element 6 with the knee joint implant augmentation 8. The first contact surface 14 of the knee joint implant augmentation 8 rests flat on the base plate 22 of the tibia element 6. The tibia shaft 26 protrudes from the second contact surface 16. The (reinforcement) wing 28 connects the tibia shaft 26 to the base plate 22 and is received in the notch 38 of the knee joint implant augmentation 8. The patellar lateral-surface portion 20 is angled with respect to a longitudinal axis 48 of the tibia shaft 26 at a closed angle not equal to 0°.

1. A knee joint implant augmentation configured to be arranged between a knee joint implant and a tibia in an implanted state, the knee joint implant augmentation comprising:
a first contact surface which, in the implanted state, is configured to rest at least partially flat on the knee joint implant;
a first rim of the first contact surface;
a second contact surface lying opposite the first contact surface and configured to rest at least partially flat on the tibia in the implanted state;
a second rim of the second contact surface; and
a lateral surface, which connects the first rim of the first contact surface to the second rim of the second contact surface,
wherein the lateral surface comprises a lateral-surface portion which is arranged in a patellar position in the implanted state of the knee joint implant augmentation, and
wherein the lateral-surface portion is angled starting from the first contact surface in the patellar direction at a closed angle greater than 0° with respect to an axis perpendicular to the first contact surface.

2. The knee joint implant augmentation according to claim 1, wherein the patellar lateral-surface portion is angled, in the implanted state of the knee joint implant augmentation, at a closed angle greater than 0° with respect to a longitudinal axis of a medullary canal of the tibia.

3. The knee joint implant augmentation according to claim 1, wherein the first contact surface points in a proximal direction in the implanted state of the knee joint implant augmentation.

4. The knee joint implant augmentation according to claim 1, wherein a closed angle between the first contact surface and the patellar lateral-surface portion in the implanted state of the knee joint implant augmentation is greater than 90°, such that the lateral-surface portion spans a nose that extends over a base area of the first contact surface.

5. The knee joint implant augmentation according to claim 1, wherein an angle between the second contact surface and the patellar lateral-surface portion in the implanted state of the knee joint implant augmentation is less than 90°.

6. The knee joint implant augmentation according to claim 1, wherein the patellar lateral-surface portion is positioned at a leg of the knee joint implant augmentation.

7. The knee joint implant augmentation according to claim 1, wherein the leg is in a patellar position in the implanted state of the knee joint implant augmentation.

8. The knee joint implant augmentation according to claim 1, wherein an elongated notch in the knee joint implant augmentation is prepared and configured to receive a reinforcing wing of the knee joint implant, and the patellar lateral-surface portion extends substantially parallel to a longitudinal extent of the notch.

9. A knee joint implant comprising:
a femur element;
a tibia element comprising a base plate, a shaft extending from the base plate, and a knee joint implant augmentation having a first contact surface, which rests at least partially flat against the base plate;
a first rim of the first contact surface;
a second contact surface arranged opposite the first contact surface and configured to be able to rest at least partially flat on the tibia in an implanted state of the knee joint implant;
a second rim of the second contact surface;
a lateral surface, which connects the first rim to the second rim; and
a lateral-surface portion of the lateral surface which is arranged in a patellar position in the implanted state of the knee joint implant,
wherein the lateral-surface portion is angled at a closed angle greater than 0° with respect to a longitudinal axis of the shaft.

10. The knee joint implant according to claim 9, wherein a closed angle between the second contact surface and the lateral-surface portion is smaller than 90°.

11. The knee joint implant according to claim 9, wherein the first contact surface at least partially corresponds to a shape of the base plate.

12. The knee joint implant according to claim 9, wherein the second contact surface is configured to correspond to an outer contour of the tibia at least partially.

13. The knee joint implant according to claim 9, wherein a closed angle between the lateral-surface portion and the first contact surface is greater than 90°, such that the lateral-surface portion spans a nose that extends over a base area of the first contact surface.

14. The knee joint implant according to claim 9, wherein the tibia element comprises a reinforcing wing extending between the base plate and the shaft.
